(19) United States
(12) Patent Application Publication
(10) Pub. No.: US 2026/0240418 A1
(43) Pub. Date: Aug. 20, 2026

(54) SURGICAL TOOL TRANSFERRING APPARATUS

(71) Applicant: ROEN SURGICAL, INC., Daejeon (KR)

(72) Inventors: Kyu-Seob SONG, Daejeon (KR); Duk-Yoo KONG, Daejeon (KR); Duk-Sang KIM, Daejeon (KR); Dong-Ho LEE, Daejeon (KR); Hyun-Woo BAEK, Daejeon (KR); Joon-Yeong KIM, Daejeon (KR); Un-Je YANG, Daejeon (KR); Joon-Hwan KIM, Daejeon (KR); Jae-Chul KIM, Daejeon (KR)

(73) Assignee: ROEN SURGICAL, INC., Daejeon (KR)

(21) Appl. No.: 19/164,547

(22) PCT Filed: Mar. 12, 2024
(86) PCT No.: PCT/KR2024/003135

§ 371 Date: Sep. 11, 2025

(30) Foreign Application Priority Data
Mar. 15, 2023 (KR) 10-2023-0034075

(51) Int. Cl. A61B 1/00; A61B 1/005; A61B 1/018

(52) U.S. Cl. CPC A61B 1/00133; A61B 1/00128; A61B 1/0051; A61B 1/018

(57) ABSTRACT

Proposed is a surgical tool transferring apparatus. The surgical tool transferring apparatus includes an overtube which is flexible enough to be inserted into a subject and which includes a tool channel and a connecting member channel, a surgical tool inserted into the tool channel, and a connecting member inserted into the connecting member channel and connected to a coupling part of the surgical tool by passing through a connection point between the tool channel and the connecting member channel. The surgical tool is moved in the tool channel such that the coupling part is positioned adjacent to the connection when the connecting member is pulled. Therefore, even when a bend occurs in the overtube, the surgical tool inserted into the tool channel of the overtube is capable of being rapidly and smoothly moved to an affected region inside the human body.

### TECHNICAL FIELD

The present disclosure relates to a surgical tool transferring apparatus. More particularly, the present disclosure relates to a surgical tool transferring apparatus configured to move a surgical tool in a tool channel of an endoscope.

### BACKGROUND ART

Upon early detection of digestive system cancer, the importance of endoscopic resection that removes the affected region with an endoscope equipped with a surgical knife is increasing. Endoscopic resection includes endoscopic mucosal resection and endoscopic submucosal dissection.

Early digestive cancer and gastrointestinal polyps which can be developed to cancer can be treated with endoscopic resection, and five-year survival rate is high close to almost 100% upon early diagnosis and treatment. Endoscopic resection has the advantage of reducing the recovery period by reducing strain on a patient's body comparing to an existing surgical method incising the skin and approaching the affected region.

The main function of the endoscope is to provide a passage (hereinafter, referred to as a “tool channel”) through which a surgical tool is inserted into a surgical site. In order to position the endoscope on a lesion, a long tube (hereinafter, referred to as an “overtube”) surrounding the surgical tool and a camera is required to have a shape which is not a straight line shape and which is capable of being bent. That is, the overtube is required to reach a desired position by being moved flexibly and passing through a passage inside the human body, and there must be no protrusion corner when the overtube is bent.

In a commercial endoscope, the surgical tool is pushed into the tool channel by a manual operation performed by a surgeon, and is moved to a distal part of the overtube. In an endoscopic surgical apparatus (see Korean Patent No. 10-2340169), a surgical tool is pushed into a tool channel by an actuator controlled by a remote master device, and is moved to a distal part of an overtube.

As the overtube is bent corresponding to a shape of a passage in the human body, the surgical tool is moved to the distal part of the overtube while friction between the surgical tool and an inner surface of the tool channel is formed. However, a force that the surgical tool is pushed into the tool channel is applied to the surgical tool at an outer side of a natural opening (hereinafter, referred to as a “first point”) such as a patient's mouth, anus, vagina, urethra and so on, while the generation of a friction force between the surgical tool and the inner surface of the tool channel is distributed in front of the first point on the basis of a progression direction of the surgical tool. Therefore, there is a problem that it is difficult to move the surgical tool smoothly within the tool channel.

A joint structure of a representative endoscopic surgical tool is driven by a wire, and each joint has a gear-shaped rolling engagement. Such a gear-shaped rolling engagement is advantageous for a stable driving, but has a disadvantage that an accurate interpretation of kinematics thereof is difficult to be performed, and miniaturization of such an endoscopic surgical tool is difficult to be realized due to the complexity of the structure.

In addition, the endoscopic surgical tool is required to have a rigidity when a tension is applied to the wire, and the surgical tool is required to be moved smoothly in the tool channel even when the overtube is bent. Therefore, in order to smoothly move the surgical tool within the tool channel, the overtube is required to have the tool channel having a sufficient size (diameter) to an end of the surgical tool.

However, the end of the overtube is generally provided with a camera, a lighting, a nozzle, and an end of the tool channel. At least one tool channel may be provided. Therefore, there is a limit to increasing the size (diameter) of the tool channel. Therefore, even when the overtube is bent, a method capable of smoothly moving the surgical tool within the tool channel having a limited size (diameter) is required.
(Patent Document 0001) Korean Patent No. 10-2379863 (registration date: Mar. 24, 2022)

### DISCLOSURE

### Technical Problem

An objective of the present disclosure is to provide a surgical tool transferring apparatus capable of rapidly and smoothly moving a surgical tool inserted into a tool channel of an overtube to an affected region inside the human body even when a bent occurs in the overtube.

### Technical Solution

According to the present disclosure, the objective of the present disclosure may be realized by a surgical tool transferring apparatus including: an overtube which is flexible enough to be inserted into a subject and which includes a tool channel and a connecting member channel; a surgical tool inserted into the tool channel; and a connecting member inserted into the connecting member channel and connected to a coupling part of the surgical tool by passing through a connection point between the tool channel and the connecting member channel, wherein the surgical tool is moved in the tool channel such that the coupling part is positioned adjacent to the connection point n the connecting member is pulled.

The surgical tool may include: a bending part configured to be bent by manipulating a manipulating device; and a flexible tube connecting the bending part and the manipulating device to each other, wherein the coupling part may be disposed on one side of the flexible tube or one side of the bending part.

The surgical tool may include: a bending part configured to be bent by manipulating a manipulating device; and a flexible tube connecting the bending part and the manipulating device to each other, wherein the coupling part may be interposed between the bending part and the flexible tube.

A connecting member fixing part that protrudes toward an inner surface of the tool channel may be formed on the coupling part, and the connecting member may be coupled to the connecting member fixing part.

The connecting member may be coupled to a connecting member fixing part, and a coupling groove into which the connecting member fixing part is inserted such that the connecting member fixing part is capable of being attached to and detached from the coupling groove may be formed in an outer surface of the coupling part.

An end portion of the connecting member may form a snare portion such that the snare portion is configured to tighten an outer surface of the coupling part by a tension of the connecting member.

A plurality of spheres may be threaded on the snare portion of the connecting member.

The coupling part may include: a coupling body tightened by the snare portion; and a boundary body positioned on both ends of the coupling body with respect to a direction in which the surgical tool is moved in the tool channel, the boundary body being positioned closer to an inner surface of the tool channel than the coupling body.

The connecting member may be coupled to a bistable band having a bistable structure, and the bistable band may be coupled to an outer surface of the coupling part such that the bistable band is capable of being attached to and detached from the outer surface of the coupling part.

The connecting member channel may include a first connecting member channel and a second connecting member channel, wherein the connection point may include: a first connection point connecting the tool channel and the first connecting member channel to each other; and a second connection point connecting the tool channel and the second connecting member channel to each other, and wherein the connecting member may include: a first connecting member inserted into the first connecting member channel and connected to the coupling part through the first connection point; and a second connecting member inserted into the second connecting member channel and connected to the coupling part through the second connection point.

The tool channel may include a first tool channel and a second tool channel, wherein the surgical tool may include: a first surgical tool inserted into the first tool channel; and a second surgical tool inserted into the second tool channel, wherein the first connection point may connect the first tool channel and the first connecting member channel to each other and the second connection point may connect the second tool channel and the second connecting member channel to each other, and wherein the coupling part may include: a first coupling part which is disposed on the first surgical tool and to which the first connecting member is connected; and a second coupling part which is disposed on the second surgical tool and to which the second connecting member is connected.

The tool channel and the connecting member channel may be opened at a tip end of the overtube, and the connection point may be provided on the tip end of the overtube.

The connection point may connect the tool channel and the connecting member channel to each other within the overtube.

A pulley may be coupled to the overtube such that the pulley is capable of being rotated, and the pulley may support the connecting member at the connection point such that the pulley is capable of being rotated.

The overtube may include: a main tube forming the tool channel; and an auxiliary tube attached to the main tube such that the connecting member channel is formed along a longitudinal direction of the main tube.

A cap may be coupled to a tip end of the main tube, and the cap may form a portion of the connection point.

The surgical tool transferring apparatus may further include a roll feeder configured to move the surgical tool by a rotational force of a rotation roller, wherein the connecting member may be interlocked with the rotation roller such that the connecting member is wound on or unwound from a rotating interlocking pulley so that a movement distance of the connecting member and a movement distance of the surgical tool in the tool channel are the same.

### Advantageous Effects

According to the present disclosure, when the connecting member is pulled outside the human body, the surgical tool is moved in the tool channel such that the coupling part is positioned adjacent to the connection point, so that the surgical tool inserted into the tool channel of the overtube is capable of being rapidly and smoothly moved to an affected region inside the human body even when a bend occurs in the overtube.

### DESCRIPTION OF DRAWINGS

FIG. 1 is a view illustrating a usage state of a surgical tool transferring apparatus according to an embodiment of the present disclosure and illustrating a state in which a surgical tool inserted into a tool channel is moved toward a second end (hereinafter, referred to as a “distal part”) of an overtube from a first end (hereinafter, referred to as a “proximal part”) of the overtube.

FIG. 2 is a view illustrating a usage state of the surgical tool transferring apparatus according an embodiment of the present disclosure and illustrating a state in which the surgical tool positioned in the distal part of the overtube is moved toward the proximal part of the overtube.

FIG. 3 is a view illustrating a usage state of the surgical tool transferring apparatus according to an embodiment of the present disclosure and illustrating a state in which the surgical tool inserted into the tool channel is moved from the proximal part of the overtube toward the distal part of the overtube by a roll feeder.

FIG. 4 is a partially enlarged view illustrating the surgical tool transferring apparatus according to an embodiment of the present disclosure and illustrating a coupling part in which a connecting member fixing part is formed.

FIG. 5 and FIG. 6 are partially enlarged views illustrating the surgical tool transferring apparatus according to an embodiment of the present disclosure and illustrating a bistable band that is coupled to an outer surface of the coupling part such that the bistable band is capable of being attached to and detached from the coupling part.

FIG. 7 and FIG. 8 are partially enlarged views illustrating the surgical tool transferring apparatus according to an embodiment of the present disclosure and illustrating a snare portion that tightens the outer surface of the coupling part by a tension of a connecting member.

FIG. 9 is an enlarged view illustrating the distal part of the overtube of the surgical tool transferring apparatus according to an embodiment of the present disclosure and illustrating the surgical tool and the connecting member.

FIG. 10 is an enlarged view illustrating the distal part of the overtube of the surgical tool transferring apparatus according to an embodiment of the present disclosure and illustrating the surgical tool, a first connecting member, and a second connecting member.

FIG. 11 is an enlarged view illustrating the distal part of the overtube of the surgical tool transferring apparatus according to an embodiment of the present disclosure and illustrating the surgical tool, the first connecting member, the second connecting member, and a third connecting member.

FIG. 12 and FIG. 13 are enlarged views illustrating the distal part of the overtube of the surgical tool transferring apparatus according to an embodiment of the present disclosure and illustrating a first surgical tool, a second surgical tool, the first connecting member, and the second connecting member.

FIG. 14 is a view illustrating the distal part of the overtube of the surgical tool transferring apparatus according to an embodiment of the present disclosure and illustrating the first surgical tool, the second surgical tool, the first connecting member, the second connecting member, the third connecting member, and a fourth connecting member.

FIG. 15 is a cross-sectional view illustrating the distal part of the overtube of the surgical tool transferring apparatus according to an embodiment of the present disclosure and illustrating a connection point that protrudes from the distal part.

FIG. 16 is a cross-sectional view illustrating the distal part of the overtube of the surgical tool transferring apparatus according to an embodiment of the present disclosure and illustrating a pulley that protrudes from the distal part.

FIG. 17 is a cross-sectional view illustrating the distal part of the overtube of the surgical tool transferring apparatus according to an embodiment of the present disclosure and illustrating the connection point inside the overtube.

FIG. 18 is a cross-sectional view illustrating the distal part of the overtube of the surgical tool transferring apparatus according to an embodiment of the present disclosure and illustrating the pulley inside the overtube.

FIG. 19 is a cross-sectional view illustrating the distal part of the overtube of the surgical tool transferring apparatus according to an embodiment of the present disclosure and illustrating the connection point inside the overtube.

FIG. 20 is a cross-sectional view illustrating the distal part of the overtube of the surgical tool transferring apparatus according to an embodiment of the present disclosure and illustrating a first pulley and a second pulley inside the overtube.

FIG. 21 is a cross-sectional view illustrating the distal part of the overtube of the surgical tool transferring apparatus according to an embodiment of the present disclosure and illustrating the pulley that protrudes from the distal part.

FIG. 22 is a cross-sectional view illustrating the distal part of the overtube of the surgical tool transferring apparatus according to an embodiment of the present disclosure and illustrating the first pulley that protrudes from the distal part, and the second pulley and a third pulley that are disposed inside the overtube.

FIG. 23 is a cross-sectional view illustrating the distal part of the overtube of the surgical tool transferring apparatus according to an embodiment of the present disclosure and illustrating a cap and the connection point of the distal part.

FIG. 24 is a transparent perspective view illustrating the distal part of the overtube of the surgical tool transferring apparatus according to an embodiment of the present disclosure and illustrating the first surgical tool, the second surgical tool, the first connecting member, and the second connecting member.

### MODE FOR INVENTION

Hereinafter, exemplary embodiments of the present disclosure will be described in detail with reference to the accompanying drawings. However, in describing the present disclosure, description of known functions or known configurations will be omitted in order to clarify the gist of the present disclosure.

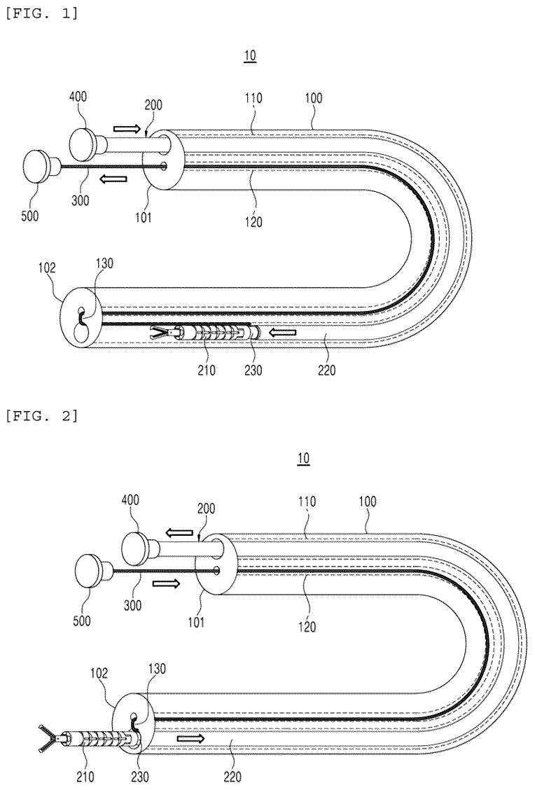

FIG. 1 is a view illustrating a usage state of a surgical tool transferring apparatus 10 according to an embodiment of the present disclosure and illustrating a state in which a surgical tool 200 inserted into a tool channel 110 is moved toward a second end 102 (hereinafter, referred to as a “distal part”) of an overtube 100 from a first end 101 (hereinafter, referred to as a “proximal part”) of the overtube 100.

FIG. 2 is a view illustrating a usage state of the surgical tool transferring apparatus 10 according to an embodiment of the present disclosure and illustrating a state in which the surgical tool 200 positioned in the distal part 102 of the overtube 100 is moved toward the proximal part 101 of the overtube 100.

As illustrated in FIG. 1 and FIG. 2, the surgical tool transferring apparatus 10 according to an embodiment of the present disclosure is configured to move the surgical tool 200 inserted into the tool channel 110 of the overtube 100 rapidly and smoothly into a subject even when the overtube 100 is bent.

Here, it should be understood that the subject refers to a human body, an animal, or other subject into which an overtube is inserted. Hereinafter, for the convenience of understanding the surgical tool transferring apparatus 10 of the present disclosure, an embodiment in which an overtube is inserted into a human body (a patient) will be described.

The surgical tool transferring apparatus 10 according to an embodiment of the present disclosure includes the overtube 100, the surgical tool 200, and a connecting member 300.

The overtube 100 has a long tube shape. The overtube 100 is a portion that enters the intraluminal space of the human body through natural openings such as the patient's mouth, anus, vagina, urethra, and so on.

The overtube 100 is inserted into the lumen and may be flexibly bent according to a curve in the lumen. The overtube 100 may be manufactured from an elastically deformable material such as medical-grade rubber, silicone, or the like.

As illustrated in FIG. 1 and FIG. 2, the overtube 100 includes the tool channel 110 and a connecting member channel 120.

The tool channel 110 forms a long passage through which the surgical tool 200 moves along a longitudinal direction of the overtube 100. The tool channel 110 may form a cylindrical passage. The tool channel 110 is opened at the proximal part 101 and the distal part 102 of the overtube 100.

The surgical tool 200 may be inserted into the tool channel 110 through an opening in the proximal part 101 outside the human body. The surgical tool 200 may access an affected region in the human body through an opening in the distal part 102 within the human body.

The connecting member channel 120 forms a long passage through which the connecting member 300 moves along the longitudinal direction of the overtube 100. The connecting member channel 120 may form a cylindrical passage.

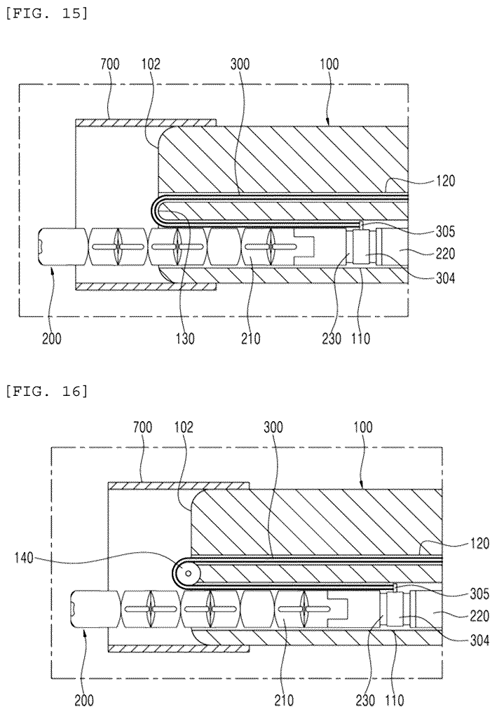

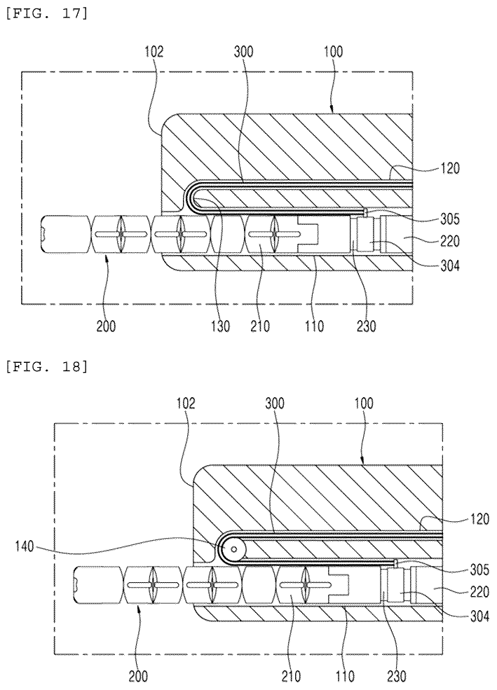

The connecting member channel 120 is opened in the proximal part 101 of the overtube 100. The connecting member channel 120 is connected to the tool channel 110 through a connection point 130 (see FIG. 15 and FIG. 17). The connection point 130 is formed on the distal part 102 of the overtube 100. The connecting member channel 120 may be directly connected to the tool channel 110.

The overtube 100 may be an insertion section of a commercial endoscope. Alternatively, as disclosed in Korean Patent No. 10-2340169, the overtube 100 may be a tube for insertion into the human body used in an endoscopic surgical apparatus.

A plurality of cables may be disposed inside the overtube 100. The cables may form a tension for bending a bending section at a distal end of the overtube 100.

As illustrated in FIG. 1 and FIG. 2, the surgical tool 200 includes an end effector, a bending part 210, and a flexible tube 220.

The end effector may be provided as forceps, a suction tube, a needle, a knife, a coagulation grasper, or the like. The end effector is illustrated as forceps in FIG. 1 and FIG. 2.

The end effector is coupled to the bending part 210. The bending part 210 forms a flexible joint structure. The bending part 210 may include a plurality of links and a plurality of tensile members.

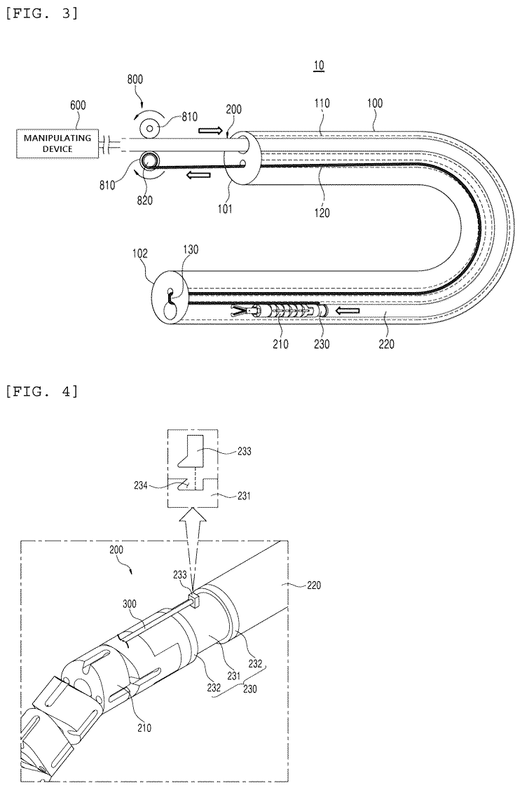

The links may be sequentially arranged from the end effector. Both ends of the tensile members may be connected to the end effector and a manipulating device 600 (see FIG. 3). The tensile members may pass through the links sequentially. When the manipulating device 600 is operated, the tensile members may be selectively pulled or released, and the bending part 210 may be bent.

As disclosed in Korean Patent No. 10-2349030, an operating structure of the tensile members and the bending part 210 is a known technology, so a detailed description thereof will be omitted.

The manipulating device 600 described above may be a driving actuator disclosed in Korean Patent No. 10-2349030.

The flexible tube 220 connects the bending part 210 and the manipulating device 600 to each other. The flexible tube 220 forms a passage that accommodates the tensile members between the bending part 210 and the manipulating device 600.

As illustrated in FIG. 1, the surgical tool 200 is inserted into the tool channel 110 outside the human body. In a process in which the surgical tool 200 is inserted into the tool channel 110, the flexible tube 220 is bent within the tool channel 110 by a curve in the lumen. The flexible tube 220 may be manufactured from an elastically deformable material such as medical-grade rubber, silicone, or the like.

In a commercial endoscope, the surgical tool 200 may be inserted into the tool channel 110 by a manual operation of a surgeon, and may be moved to the distal part 102 of the overtube 100.

Alternatively, in an endoscopic surgical apparatus (see Korean Patent No. 10-2340169), the surgical tool 200 may be inserted into the tool channel 110 by an actuator controlled by a remote master device, and may be moved to the distal part 102 of the overtube 100.

In FIG. 1 and FIG. 2, it is illustrated that the surgical tool 200 is moved to the distal part 102 of the overtube 100 by a first actuator 400 (see FIG. 1) or the surgical tool 200 is moved to the proximal part 101 of the overtube 100 by the first actuator 400 (see FIG. 2). The first actuator 400 may be provided as a linear actuator or the like.

As illustrated in FIG. 1 and FIG. 2, the connecting member 300 may be pulled or released by a second actuator 500. The second actuator 500 may be provided as a linear actuator, a step motor, or the like.

The connecting member 300 is inserted into the connecting member channel 120 from the proximal part 101 of the overtube 100, and is connected to the coupling part 230 of the surgical tool 200 by passing through the connection point 130.

The coupling part 230 refers to a portion in which the connecting member 300 is connected to the surgical tool 200. The coupling part 230 may be interposed between the bending part 210 and the flexible tube 220.

Alternatively, the coupling part 230 may be a base portion of the bending part 210. A base of the bending part 210 is a portion coupled to the flexible tube 220, and may not be rotated with respect to the flexible tube 220. Alternatively, the base of the bending part 210 may be rotated with respect to the flexible tube 220.

As illustrated in FIG. 1, when the first actuator 400 moves the surgical tool 200 inserted into the tool channel 110 from the proximal part 101 of the overtube 100 to the distal part 102 of the overtube 100, the second actuator 500 may pull the connecting member 300 inserted into the connecting member channel 120 from the proximal part 101 of the overtube 100.

The overtube 100 is moved flexibly through a curved passage in the human body, and reaches a desired position (an affected region). Therefore, the overtube 100 inserted into the lumen forms a plurality of curved portions (hereinafter, referred to as “curved parts”).

At this time, when the surgical tool 200 inserted into the tool channel 110 is moved to the distal part 102 of the overtube 100 from the proximal part 101 of the overtube 100 by the first actuator 400 only, the surgical tool 200 may form a large frictional force with an inner surface of the tool channel 110 in the curved parts. Therefore, there is a situation in which it is difficult to move the surgical tool 200.

In the surgical tool transferring apparatus 10 according to an embodiment of the present disclosure, as the second actuator 500 pulls the connecting member 300 inserted into the connecting member channel 120 from the proximal part 101 of the overtube 100, the surgical tool 200 inserted into the tool channel 110 may be smoothly moved to the distal part 102 of the overtube 100 even when the plurality of bends is formed in the overtube 100.

Alternatively, in a commercial endoscope, a surgeon may pull the connecting member 300 inserted into the connecting member channel 120 from the proximal part 101 of the overtube 100.

At this time, even if the surgeon does not push the surgical tool 200 inserted into the tool channel 110 from the proximal part 101 to the distal part 102 of the overtube 100, the surgical tool 200 may be smoothly moved to the distal part 102 of the overtube 100 by the connecting member 300.

The connection point 130 refers to a portion connecting the tool channel 110 and the connecting member channel 120 to each other. Therefore, when the second actuator 500 pulls the connecting member 300 inserted into the connecting member channel 120 from the proximal part 101 of the overtube 100, the surgical tool 200 in the tool channel 110 is moved so that the coupling part 230 is positioned adjacent to the connection point 130.

As illustrated in FIG. 1, the connection point 130 is formed on the distal part 102 of the overtube 100 (see FIG. 15 and FIG. 17). Therefore, when the second actuator 500 pulls the connecting member 300 inserted into the connecting member channel 120 from the proximal part 101 of the overtube 100, the surgical tool 200 in the tool channel 110 is moved toward the distal part 102 of the overtube 100.

As illustrated in FIG. 2, by pulling the connecting member 300, the surgical tool 200 in the tool channel is moved toward the distal part 102 of the overtube 100 until the coupling part 230 is positioned next to the connection point 130.

In this state, while the surgeon observes the affected region through a monitor connected to a camera of the surgical tool, the surgeon may operate the manipulating device 600 and treat the affected region with the surgical tool 200.

As illustrated in FIG. 2, when the surgical tool 200 is pulled out or replaced, the first actuator 400 may move the surgical tool 200 inserted in the tool channel 110 toward the proximal part 101 of the overtube 100. At this time, the second actuator 500 may release the connecting member 300 toward the tool channel 110 from the proximal part 101 side of the overtube 100.

Alternatively, when the first actuator 400 moves the surgical tool 200 inserted into the tool channel 110 toward the proximal part 101 of the overtube 100, a motor of the second actuator 500 becomes unlocked, and the connecting member 300 may be freely released toward the tool channel 110.

Alternatively, in a commercial endoscope, the surgeon may pull the surgical tool 200 inserted into the tool channel 110 from the proximal part 101 of the overtube 100. At this time, the surgeon may not apply a restraining force to the connecting member 300 inserted into the connecting member channel 120 from the proximal part 101.

Therefore, when the surgical tool 200 inserted into the tool channel 110 is pulled from the proximal part 101 of the overtube 100 by the first actuator 400 or the surgeon, the surgical tool 200 in the tool channel 110 is moved toward the proximal part 101 of the overtube 100.

At this time, the connecting member 300 is moved from the tool channel 110 to the proximal part 101 of the overtube 100 along the surgical tool 200.

The connecting member 300 may be formed of a fishing line, a wire, a thin plate material, a hydraulic tube, or the like. The connecting member 300 may pull the surgical tool 200 by tension. The connecting member 300 may be referred to as a tension member.

As an example, in a situation in which the connecting member 300 is formed of nylon thread or the like, which is a main material of the fishing line, since a surface of the nylon thread has a smooth surface, the friction of the nylon thread with the connecting member channel 120 and the tool channel 110 is small. In addition, the nylon thread is thin yet strong, so that the connecting member channel 120 may have a small diameter. In addition, since the nylon thread has durability, the nylon thread may be used for a large number of endoscopic surgeries.

FIG. 3 is a view illustrating a usage state of the surgical tool transferring apparatus 10 according to an embodiment of the present disclosure and illustrating a state in which the surgical tool 200 inserted into the tool channel 110 is moved from the proximal part 101 of the overtube 100 toward the distal part 102 of the overtube 100 by a roll feeder 800.

As illustrated in FIG. 3, the surgical tool 200 may be moved to the distal part 102 of the overtube 100 or may be moved to the proximal part 101 of the overtube 100 by the roll feeder 800. The roll feeder 800 includes a motor, a rotation roller 810, and an interlocking pulley 820.

The rotation roller 810 may be rotated in both directions by a step motor. The rotation roller 810 is provided as a pair of rotation rollers 810. The pair of rotation rollers 810 is in contact with the surgical tool 200 from opposite sides.

A rotational force of the rotation rollers 810 is converted into a motion of the surgical tool 200 by a frictional force between the rotation rollers 810 and the surgical tool 200. Therefore, the roll feeder 800 moves the surgical tool 200 by the rotational force of the rotation rollers 810.

The interlocking pulley 820 is rotated in conjunction with the rotation rollers 810. The interlocking pulley 820 may be coupled to the same shaft as one of the pair of rotation rollers 810. The connecting member 300 is mounted on the interlocking pulley 820.

As illustrated in FIG. 3, when the rotation rollers 810 move the surgical tool 200 inserted into the tool channel 110 from the proximal part 101 of the overtube 100 toward the distal part 102 of the overtube 100, the interlocking pulley 820 winds the connecting member 300 inserted into the connecting member channel 120 from the proximal part 101 of the overtube 100.

At this time, a distance that the rotating rollers 810 move the surgical tool 200 and a length of the connecting member 300 that unwound from or wound onto the interlocking pulley 820 are designed to be identical so that a movement distance of the surgical tool 200 and a movement distance of the connecting member 300 are the same. This can be achieved through diameter design of the rotation rollers 810 and the interlocking pulley 820.

FIG. 4 is a partially enlarged view illustrating the surgical tool transferring apparatus 10 according to an embodiment of the present disclosure and illustrating the coupling part 230 in which a connecting member fixing part 233 is formed. The base of the bending part 210 may not be rotated with respect to the flexible tube 220.

Alternatively, the base of the bending part 210 may be rotated with respect to the flexible tube 220. In this situation, the coupling part 230 may be coupled to the flexible tube 220. Therefore, even when the bending part 210 is rotated while the flexible tube 220 and the coupling part 230 are stationary within the tool channel 110, the bending part 210 may be freely rotated without interference (twisting) of the connecting member 300.

As illustrated in FIG. 4, the coupling part 230 includes a coupling body 231 and a boundary body 232.

The coupling body 231 has an outer surface in a cylindrical shape. The outer surface of the coupling body 231 has a diameter smaller than diameters of outer surfaces of the bending part 210 and the flexible tube 220.

The boundary body 232 is positioned at both ends of the coupling body 231 with respect to a direction in which the surgical tool 200 is moved in the tool channel 110. The boundary body 232 has an outer surface in a cylindrical shape.

The outer surface of the boundary body 232 has a diameter larger than the diameter of the outer surface of the coupling body 231. Therefore, the outer surface of the boundary body 232 is positioned closer to an inner surface of the tool channel 110 than the outer surface of the coupling body 231. The outer surface of the boundary body 232 may have the same shape as the outer surface of the bending part 210 and the outer surface of the flexible tube 220.

As illustrated in FIG. 4, the connecting member fixing part 233 that protrudes toward the inner surface of the tool channel 110 may be formed on the coupling part 230. The connecting member fixing part 233 may have a polygonal column shape such as a rectangular column shape. The connecting member fixing part 233 may have a cylindrical shape.

The connecting member 300 may be coupled to the connecting member fixing part 233. an end portion of the connecting member 300 may be coupled to the connecting member fixing part 233 by adhesion, thermal fusion, bolt fastening, and so on.

The connecting member fixing part 233 protrudes from the outer surface of the coupling body 231. Therefore, the connecting member 300 coupled to the connecting member fixing part 233 may maintain a state in which the connecting member 200 is generally parallel to the tool channel 110.

Therefore, when the connecting member 300 is pulled, friction between the connecting member 300 and the bending part 210 may be relieved. Therefore, damage to the connecting member 300 due to friction between the connecting member 300 and the bending part 210 may be suppressed.

As illustrated in the partially enlarged view in FIG. 4, a coupling groove 234 may be formed on the outer surface of the coupling part 230. A portion that protrudes to one side or both sides may be formed on a lower portion of the connecting member fixing part 233, and the lower portion of the connecting member fixing part 233 may be fitted and coupled to the coupling groove 234.

Therefore, the connecting member fixing part 233 may be inserted into the coupling groove 234 such that the connecting member fixing part 233 is capable of being attached to and detached from the coupling groove 234. The coupling structure of the connecting member fixing part 233 and the coupling groove 234 may vary, such as interference fit coupling, magnetic coupling, threaded coupling, and so on.

The connecting member 300 is coupled to the connecting member fixing part 233. The coupling groove 234 is formed on the coupling part 230 of the surgical tools 200 having different end effectors such as forceps, a suction tube, a needle, a knife, a coagulation grasper, and so on. Therefore, it is easy to replace the surgical tool 200, and the connecting member 300 may be reused even when the surgical tool 200 is replaced.

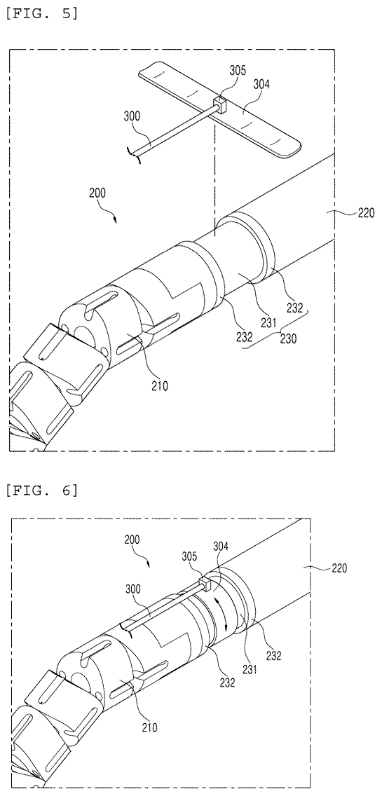

FIG. 5 and FIG. 6 are partially enlarged views illustrating the surgical tool transferring apparatus 10 according to an embodiment of the present disclosure and illustrating a bistable band 304 that is coupled to the outer surface of the coupling part 230 such that the bistable band 304 is capable of being attached to and detached from the coupling part 230.

The connecting member fixing part 305 may be disposed on the bistable band 304 having a bistable structure. The connecting member 300 is coupled to the connecting member fixing part 305, and is coupled to the bistable band 304. The bistable band 304 forms a long band shape in a longitudinal direction thereof. The bistable band 304 may be formed of metal, plastic, and so on.

The bistable band 304 has the bistable structure. In the bistable band 304, a stable structure is formed in a state in which the longitudinal direction of the bistable band 304 forms a straight line in one direction (hereinafter, a “first stable state”) and in a state in which the longitudinal direction of the bistable band 304 forms a curvature in a circumferential direction of the coupling part 230 (hereinafter, a “second stable state”).

That is, the bistable band 304 is elastically deformed in different directions in the first stable state and the second stable state. Therefore, the bistable band 304 is coupled to the outer surface of the coupling part 230 such that the bistable band 304 is capable of being attached to and detached from the outer surface of the coupling part 230. Therefore, it is easy to replace the surgical tool 200, and the connecting member 300 may be reused even when the surgical tool 200 is replaced.

The bistable band 304 may be slid in the circumferential direction on the outer surface of the coupling body 231. Therefore, even when the surgical tool 200 is rotated within the tool channel 110, entanglement between the connecting member 300 and the surgical tool 200 within the tool channel 110 is prevented.

The outer surface of the boundary body 232 has the diameter larger than the diameter of the outer surface of the coupling body 231. Therefore, the bistable band 304 is not moved toward the bending part 210 and the flexible tube 220 in a state in which the bistable band 304 is detachably coupled to the outer surface of the coupling body 231.

In FIG. 6, reference numeral 304 may refer to a rotating body mounted on the outer surface of the coupling part 230 such that the rotating body is capable of being rotated. As an example, the rotating body may be provided as a bearing and so on. That is, the rotating body may not be detached from the outer surface of the coupling part 230 but may be maintained in a state in which the rotating body is mounted on the outer surface of the coupling member 230 and the rotating body is capable of being rotated.

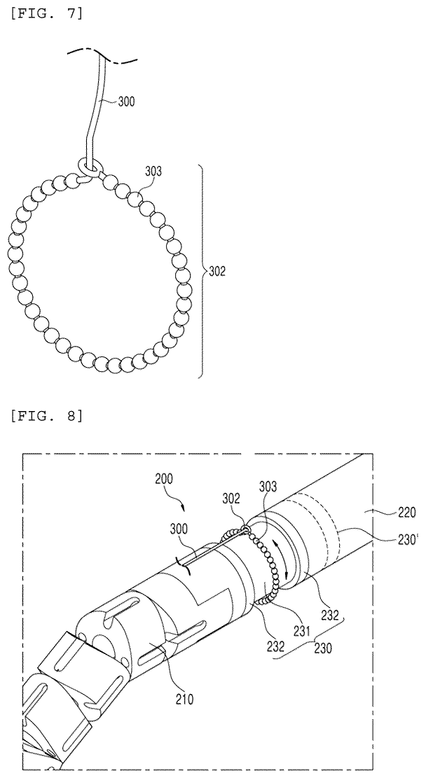

FIG. 7 and FIG. 8 are partially enlarged views illustrating the surgical tool transferring apparatus 10 according to an embodiment of the present disclosure and illustrating a snare portion 302 that tightens the outer surface of the coupling part 230 by the tension of the connecting member 300.

As illustrated in FIG. 7, the end portion of the connecting member 300 may form a snare so that the outer surface of the coupling part 230 is tightened by the tension of the connecting member 300.

The snare portion 302 is connected to the coupling part 230 in a form that tightens the outer surface of the coupling body 231. The outer surface of the boundary body 232 has the diameter larger than the diameter of the outer surface of the coupling body 231. Therefore, the snare portion 302 is not moved toward the bending part 210 and the flexible tube 220 while the snare portion 302 tightens the outer surface of the coupling body 231.

As illustrated in FIG. 1, when the connecting member 300 pulls the surgical tool 200, the snare portion 302 further tightens the outer surface of the coupling body 231 by the tension of the connecting member 300. Therefore, when the connecting member 300 pulls the surgical tool 200, the connection structure between the connecting member 300 and the coupling part 230 may be maintained rigidly.

As illustrated in FIG. 2, even when the connecting member 300 is pulled by the surgical tool 200, the snare portion 302: tightens the outer surface of the coupling body 231 by the tension of the connecting member 300. Therefore, even when the connecting member 300 is pulled by the surgical tool 200, the connection structure between the connecting member 300 and the coupling part 230 may be maintained rigidly.

The snare portion 302 may slide in the circumferential direction on the outer surface of the coupling body 231. Therefore, even when the surgical tool 200 is rotated within the tool channel 110, entanglement between the connecting member 300 and the surgical tool 200 within the tool channel 110 is prevented.

A plurality of spheres 303 may be threaded onto the connecting member 300 at the snare portion 302. The plurality of spheres 303 may be formed of metal or plastic. The plurality of spheres 303 is threaded on the connecting member 300 such that the plurality of spheres 303 is capable of being rotated.

Therefore, the snare portion 302 may easily slide in the circumferential direction on the outer surface of the coupling body 231. Therefore, even when the surgical tool 200 is rotated within the tool channel 110, entanglement between the connecting member 300 and the surgical tool 200 within the tool channel 110 is prevented.

As illustrated in FIG. 8, a coupling part 230′ may be disposed on one side of the flexible tube 220. The dotted line in FIG. 8 refers to the coupling part 230′ on which the connecting member 300 is capable of being wound. The coupling part 230′ may be formed as a groove into which the snare portion 302 is inserted.

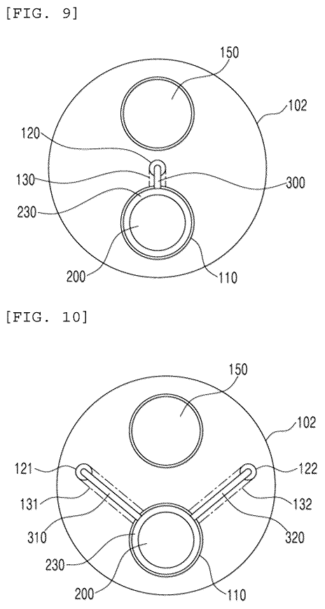

FIG. 9 is an enlarged view illustrating the distal part 102 of the overtube 100 of the surgical tool transferring apparatus 10 according to an embodiment of the present disclosure and illustrating the surgical tool 200 and the connecting member 300.

A camera 150 and a distal end of the tool channel 110 is disposed on the distal part 102 of the overtube 100. Of course, a lighting, a nozzle, and so on may be further disposed on the distal part 102 of the overtube 100.

As illustrated in FIG. 9, the tool channel 110 and the connecting member channel 120 may be opened at a tip end (distal part 102) of the overtube 100, and the connection point 130 may be disposed at a tip end of the overtube 100.

Therefore, the connecting member channel 120 is connected to the tool channel 110 through the connection point 130 at the distal part 102 of the overtube 100. Therefore, the connecting member 300 passes through the connection point 130 from the connecting member channel 120 and is connected to the coupling part 230 of the surgical tool 200.

When the connecting member 300 is pulled, a friction force between the connecting member 300 and an inner surface of the connecting member channel 120 may act on the overtube 100. The connecting member channel 120 may be positioned at the center of the overtube 100. Therefore, eccentricity of the overtube 100 due to the friction force between the connecting member 300 and the inner surface of the connecting member channel 120 may be relieved.

FIG. 10 is an enlarged view illustrating the distal part 102 of the overtube 100 of the surgical tool transferring apparatus 10 according to an embodiment of the present disclosure and illustrating the surgical tool 200, a first connecting member 310, and a second connecting member 320.

As illustrated in FIG. 10, the connecting member 300 may include the first connecting member 310 and the second connecting member 320, and the connecting member channel 120 may include a first connecting member channel 121 and a second connecting member channel 122.

The connection point 130 may include a first connection point 131 and a second connection point 132. The first connection point 131 connects the tool channel 110 and the first connecting member channel 121 to each other. The second connection point 132 connects the tool channel 110 and the second connecting member channel 122 to each other.

Therefore, the first connecting member 310 is inserted into the first connecting member channel 121, and is connected to the coupling part 230 through the first connection point 131. The second connecting member 320 is inserted into the second connecting member channel 122, and is connected to the coupling part 230 through the second connection point 132.

The first connecting member 310 and the second connecting member 320 may be pulled or released together by the second actuator 500. When the first connecting member 310 and the second connecting member 320 are pulled, a friction force between the first connecting member 310 and an inner surface of the first connecting member channel 121 and a friction force between the second connecting member 320 and an inner surface of the second connecting member channel 122 may act on the overtube 100.

The first connecting member channel 121 and the second connecting member channel 122 may be symmetrical with respect to an imaginary line connecting the center of the tool channel 110 and the center of the overtube 100.

Therefore, eccentricity of the overtube 100 due to the friction force between the first connecting member 310 and the inner surface of the first connecting member channel 121 and the friction force between the second connecting member 320 and the inner surface of the second connecting member channel 122 may be relieved.

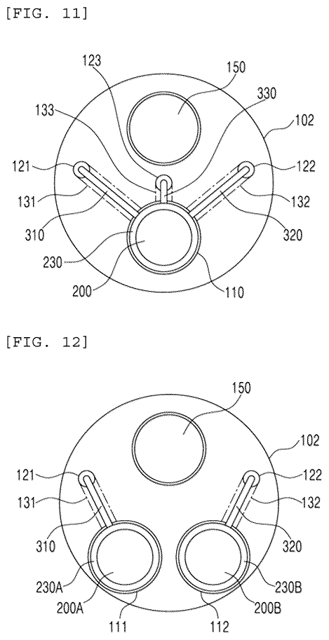

FIG. 11 is an enlarged view illustrating the distal part 102 of the overtube 100 of the surgical tool transferring apparatus 10 according to an embodiment of the present disclosure and illustrating the surgical tool 200, the first connecting member 310, the second connecting member 320, and a third connecting member 330.

As illustrated in FIG. 11, the connecting member 300 may include the first connecting member 310, the second connecting member 320, and the third connecting member 330, and the connecting member channel 120 may include the first connecting member channel 121, the second connecting member channel 122, and a third connecting member channel 123.

The connection point 130 may include the first connection point 131, the second connection point 132, and a third connection point 133. The first connection point 131 connects the tool channel 110 and the first connecting member channel 121 to each other. The second connection point 132 connects the tool channel 110 and the second connecting member channel 122 to each other. The third connection point 133 connects the tool channel 110 and the third connecting member channel 123 to each other.

The first connecting member 310 is inserted into the first connecting member channel 121, and is connected to the coupling part 230 through the first connection point 131. The second connecting member 320 is inserted into the second connecting member channel 122, and is connected to the coupling part 230 through the second connection point 132. The third connecting member 330 is inserted into the third connecting member channel 123, and is connected to the coupling part 230 through the third connection point 133.

The first connecting member 310, the second connecting member 320, and the third connecting member 330 may be pulled or released together by the second actuator 500.

When the first connecting member 310, the second connecting member 320, and the third connecting member 330 are pulled, the friction force between the first connecting member 310 and the inner surface of the first connecting member channel 121, the friction force between the second connecting member 320 and the inner surface of the second connecting member channel 122, and a friction force between the third connecting member 330 and an inner surface of the third connecting member channel 123 may act on the overtube 100.

The third connecting member channel 123 may be positioned at the center of the overtube 100 . . . . In addition, the first connecting member channel 121 and the second connecting member channel 122 may be symmetrical with respect to an imaginary line connecting the center of the tool channel 110 and the center of the overtube 100.

Therefore, eccentricity of the overtube 100 due to the friction force between the first connecting member 310 and the inner surface of the first connecting member channel 121, the friction force between the second connecting member 320 and the inner surface of the second connecting member channel 122, and the friction force between the third connecting member 330 and the inner surface of the third connecting member channel 123 may be relieved.

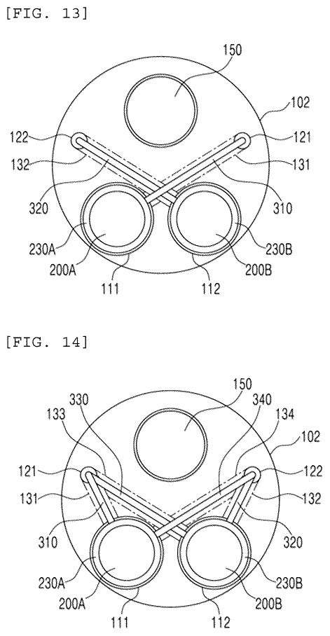

FIG. 12 and FIG. 13 are enlarged views illustrating the distal part 102 of the overtube 100 of the surgical tool transferring apparatus 10 according to an embodiment of the present disclosure and illustrating a first surgical tool 200A, a second surgical tool 200B, the first connecting member 310, and the second connecting member 320.

As illustrated in FIG. 12, the surgical tool 200 may include the first surgical tool 200A and the second surgical tool 200B, and the tool channel 110 may include a first tool channel 111 and a second tool channel 112. The first surgical tool 200A is inserted into the first tool channel 111, and the second surgical tool 200B is inserted into the second tool channel 112.

The connecting member 300 may include the first connecting member 310 and the second connecting member 320, and the connecting member channel 120 may include the first connecting member channel 121 and the second connecting member channel 122.

The connection point 130 may include the first connection point 131 and the second connection point 132. The first connection point 131 connects the first tool channel 111 and the first connecting member channel 121 to each other. The second connection point 132 connects the second tool channel 112 and the second connecting member channel 122 to each other.

The coupling part 230 may include a first coupling part 230A and a second coupling part 230B. The first coupling part 230A is disposed on the first surgical tool 200A, and the first connecting member 310 is connected to the first coupling part 230A. The second coupling part 230B is disposed on the second surgical tool 200B, and the second coupling member 320 is connected to the second coupling part 230B.

Therefore, the first connecting member 310 is inserted into the first connecting member channel 121, and is connected to the first coupling part 230A through the first connection point 131. The second connecting member 320 is inserted into the second connecting member channel 122, and is connected to the second coupling part 230B through the second connection point 132.

The first connecting member 310 and the second connecting member 320 may be pulled or released together by the second actuator 500. When the first connecting member 310 and the second connecting member 320 are pulled, the friction force between the first connecting member 310 and the inner surface of the first connecting member channel 121 and the friction force between the second connecting member 320 and the inner surface of the second connecting member channel 122 may act on the overtube 100.

On the basis of an imaginary line connecting a center portion between the first tool channel 111 and the second tool channel 112 and the center of the overtube 100, the first tool channel 111 and the second tool channel 112 may be symmetrical to each other, the first connecting member channel 121 and the second connecting member channel 122 may be symmetrical to each other, and the first connection point 131 and the second connection point 132 may be symmetrical to each other.

Therefore, eccentricity of the overtube 100 due to the friction force between the first connecting member 310 and the inner surface of the first connecting member channel 121 and the friction force between the second connecting member 320 and the inner surface of the second connecting member channel 122 may be relieved.

As illustrated in FIG. 13, the positions of the first connecting member channel 121 and the second connecting member channel 122 may be interchanged. At this time, the first connection point 131 and the second connection point 132 may be formed in shapes intersecting each other.

In order to prevent friction between the first connecting member 310 and the second connecting member 320, the first connection point 131 and the second connection point 132 may have a step difference at a portion where the first connecting member 310 and the second connecting member 320 are intersecting each other. As an example, the first connection point 131 may protrude in the form of a bridge at the intersecting portion, and the second connection point 132 may be positioned below the first connection point 131.

FIG. 14 is a view illustrating the distal part 102 of the overtube 100 of the surgical tool transferring apparatus 10 according to an embodiment of the present disclosure and illustrating the first surgical tool 200A, the second surgical tool 200B, the first connecting member 310, the second connecting member 320, the third connecting member 330, and a fourth connecting member 340.

As illustrated in FIG. 14, the surgical tool 200 may include the first surgical tool 200A and the second surgical tool 200B, and the tool channel 110 may include the first tool channel 111 and the second tool channel 112. The first surgical tool 200A is inserted into the first tool channel 111, and the second surgical tool 200B is inserted into the second tool channel 112.

The connecting member 300 may include the first connecting member 310, the second connecting member 320, the third connecting member 330, and the fourth connecting member 340, and the connecting member channel 120 may include the first connecting member channel 121 and the second connecting member channel 122.

The connection point 130 may include the first connection point 131, the second connection point 132, the third connection point 133, and a fourth connection point 134.

The first connection point 131 connects the first tool channel 111 and the first connecting member channel 121 to each other. The second connection point 132 connects the second tool channel 112 and the second connecting member channel 122 to each other. The third connection point 133 connects the second tool channel 112 and the first connecting member channel 121 to each other.

The fourth connection point 134 connects the first tool channel 111 and the second connecting member channel 122 to each other.

The coupling part 230 may include the first coupling part 230A and the second coupling part 230B. The first coupling part 230A is disposed on the first surgical tool 200A, and the first connecting member 310 and the fourth connecting member 340 are connected to the first coupling part 230A. The second coupling part 230B is disposed on the second surgical tool 200B, and the second coupling member 320 and the third coupling member 330 are connected to the second coupling part 230B.

Therefore, the first connecting member 310 is inserted into the first connecting member m channel 121, and is connected to the first coupling part 230A through the first connection point 131. The second connecting member 320 is inserted into the second connecting member channel 122, and is connected to the second coupling part 230B through the second connection point 132.

The third connecting member 330 is inserted into the first connecting member channel 121, and is connected to the second coupling part 230B through the third connection point 133. The fourth connecting member 340 is inserted into the second connecting member channel 122, and is connected to the first coupling part 230A through the fourth connection point 134.

The first connecting member 310, the second connecting member 320, the third connecting member 330, and the fourth connecting member 340 may be pulled or released together by the second actuator 500.

When the first connecting member 310, the second connecting member 320, the third connecting member 330, and the fourth connecting member 340 are pulled, the friction force between the first connecting member 310 and the inner surface of the first connecting member channel 121, the friction force between the second connecting member 320 and the inner surface of the second connecting member channel 122, a friction force between the third connecting member 330 and the inner surface of the first connecting member channel 121, and a friction force between the fourth connecting member 340 and the inner surface of the second connecting member channel 122 may act on the overtube 100.

On the basis of the imaginary line connecting the center portion between the first tool channel 111 and the second tool channel 112 and the center of the overtube 100, the first tool channel 111 and the second tool channel 112 may be symmetrical to each other, the first connecting member channel 121 and the second connecting member channel 122 may be symmetrical to each other, the first connection point 131 and the second connection point 132 may be symmetrical to each other, and the third connection point 133 and the fourth connection point 134 may be symmetrical to each other.

Therefore, eccentricity of the overtube 100 due to the friction force between the first connecting member 310 and the inner surface of the first connecting member channel 121, the friction force between the second connecting member 320 and the inner surface of the second connecting member channel 122, the friction force between the third connecting member 330 and the inner surface of the first connecting member channel 121, and the friction force between the fourth connecting member 340 and the inner surface of the second connecting member channel 122 may be relieved.

FIG. 15 is a cross-sectional view illustrating the distal part 102 of the overtube 100 of the surgical tool transferring apparatus 10 according to an embodiment of the present disclosure and illustrating the connection point 130 that protrudes from the distal part 102. FIG. 15 may be a cross-sectional view illustrating the tool channel 110 and the connecting member channel 120 in FIG. 9.

As illustrated in FIG. 15, the connection point 130 may protrude further from the distal part 102 of the overtube 100 than the tool channel 110 and the connecting member channel 120.

A cap 700 may be coupled to the tip end of the overtube 100. The cap 700 protrudes further forward than the distal part 102 of the overtube 100. The cap 700 may be formed of a flexible material capable of being flattened when the cap 700 is in contact with the internal mucous membrane of the organ. As an example, the cap 700 may be manufactured from an elastically deformable material such as medical-grade rubber, silicone, or the like.

Since the cap 700 is flattened when the cap 700 is in contact with the internal mucous membrane of the organ, a scratching phenomenon of the mucous membrane that may occur when the overtube 100 having a large diameter is inserted into the esophagus and the large intestine may be mitigated. In addition, the cap 700 may block direct contact between the connection point 130 and the internal mucous membrane of the organ.

FIG. 16 is a cross-sectional view illustrating the distal part 102 of the overtube 100 of the surgical tool transferring apparatus 10 according to an embodiment of the present disclosure and illustrating a pulley 140 that protrudes from the distal part 102.

When the connecting member 300 is pulled, the connecting member 300 is moved in opposite directions in the connecting member channel 120 and in the tool channel 110. At this time, the movement direction of the connecting member 300 is changed at the connection point 130. Therefore, the friction force between the connecting member 300 and the flexible tube 220 may be maximized at the connection point 130.

As illustrated in FIG. 16, the pulley 140 is coupled to the overtube 100 such that the pulley 140 is capable of being rotated, and the pulley 140 may support the connecting member 300 at the connection point 130 such that the connecting member 300 is capable of being rotated.

Therefore, since the friction force of the connecting member 300 at the connection point is relieved by the pulley 140, an energy required for driving the second actuator 500 may be reduced. In the commercial endoscope, the surgeon may pull the connecting member 300 with a small force.

In addition, since the friction force of the connecting member 300 at the connection point is relieved by the pulley 140, the shape deformation of the overtube 100 due to friction between the connecting member 300 and the overtube 100 may be mitigated.

FIG. 17 is a cross-sectional view illustrating the distal part 102 of the overtube 100 of the surgical tool transferring apparatus 10 according to an embodiment of the present disclosure and illustrating the connection point 130 inside the overtube 100.

As illustrated in FIG. 17, the connection point 130 may connect the tool channel 110 and the connecting member channel 120 to each other within the overtube 100. Therefore, contamination of the connecting member 300 and the connecting member channel 120 by various substances such as mucus, blood, and so on in the lumen may be prevented.

FIG. 18 is a cross-sectional view illustrating the distal part 102 of the overtube 100 of the surgical tool transferring apparatus 10 according to an embodiment of the present disclosure and illustrating the pulley 140 inside the overtube 100.

As illustrated in FIG. 18, the pulley 140 is coupled to an inner portion of the overtube 100 such that the pulley 140 is capable of being rotated, and the pulley 140 may support the connecting member 300 within the connection point 130 such that the connecting member 300 is capable of being rotated.

Therefore, since the friction force of the connecting member 300 at the connection point 130 is relieved, an energy required for driving the second actuator 500 may be reduced. In the commercial endoscope, the surgeon may pull the connecting member 300 with a small force.

In addition, since the friction force of the connecting member 300 at the connection point 130 is relieved, the shape deformation of the overtube 100 due to friction between the connecting member 300 and the overtube 100 may be mitigated.

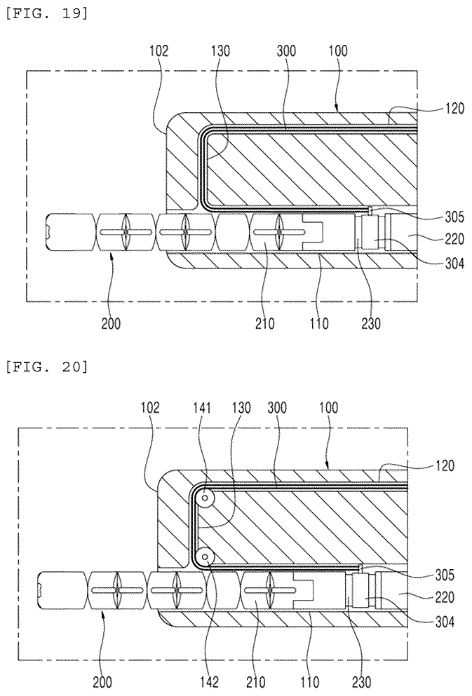

FIG. 19 is a cross-sectional view illustrating the distal part 102 of the overtube 100 of the surgical tool transferring apparatus 10 according to an embodiment of the present disclosure and illustrating the connection point 130 inside the overtube 100.

A gap between the tool channel 110 and the connecting member channel 120 illustrated in FIG. 19 may be larger than a gap between the tool channel 110 and the connecting member channel 120 illustrated in FIG. 17.

The gap between the tool channel 110 and the connecting member channel 120 may be determined according to positions of the camera 150, the lighting, the nozzle, and so on disposed on the distal part 102 of the overtube 100. In addition, the gap between the tool channel 110 and the connecting member channel 120 may be determined according to the number of the tool channels 110 and the number of the connecting member channels 120.

FIG. 20 is a cross-sectional view illustrating the distal part 102 of the overtube 100 of the surgical tool transferring apparatus 10 according to an embodiment of the present disclosure and illustrating a first pulley 141 and a second pulley 142 inside the overtube 100.

When the connecting member 300 is pulled, the connecting member 300 is moved in opposite directions in the connecting member channel 120 and in the tool channel 110. The movement direction of the connecting member 300 is changed at the connection point 130, and the friction force between the connecting member 300 and the flexible tube 220 may be maximized at the connection point 130.

As illustrated in FIG. 20, the first pulley 141 and the second pulley 142 are coupled to the inner portion of the overtube 100 such that the first pulley 141 and the second pulley 142 are capable of being rotated, and are disposed at positions where the movement direction of the connecting member 300 is changed at the connection point 130, thereby being capable of supporting the connecting member 300 such that the first pulley 141 and the second pulley 142 are capable of being rotated.

Therefore, since the friction force of the connecting member 300 at the connection point 130 is relieved, the energy required for driving the second actuator 500 may be reduced. In the commercial endoscope, the surgeon may pull the connecting member 300 with a small force.

In addition, since the friction force of the connecting member 300 at the connection point 130 is relieved, the shape deformation of the overtube 100 due to friction between the connecting member 300 and the overtube 100 may be mitigated.

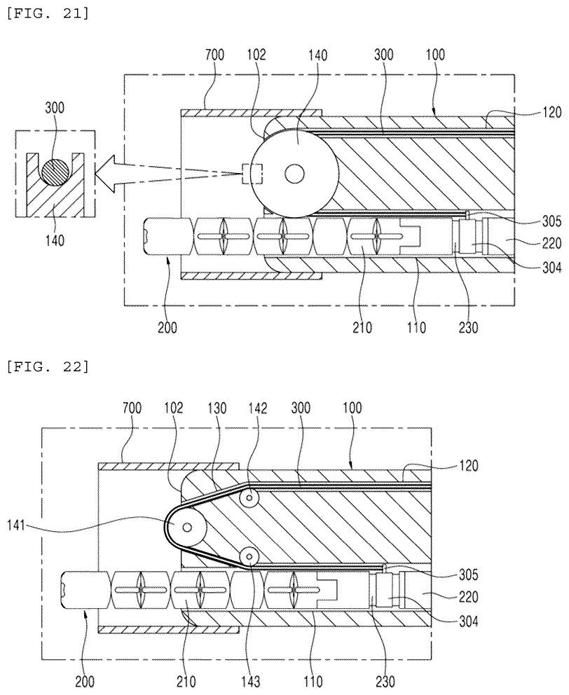

FIG. 21 is a cross-sectional view illustrating the distal part 102 of the overtube 100 of the surgical tool transferring apparatus 10 according to an embodiment of the present disclosure and illustrating the pulley 140 that protrudes from the distal part 102.

A gap between the tool channel 110 and the connecting member channel 120 illustrated in FIG. 21 may be larger than a gap between the tool channel 110 and the connecting member channel 120 illustrated in FIG. 16. In addition, the connection point 130 may form a constant curvature between the connecting member channel 120 and the tool channel 110.

The single pulley 140 is coupled to the overtube 100 such that the pulley 140 is capable of being rotated 140, and the pulley 140 may support the connecting member 300 throughout the connection point 130 such that the connecting member 300 is capable of being rotated.

Therefore, since the friction force of the connecting member 300 at the connection point 130 is relieved, the energy required for driving the second actuator 500 may be reduced. In the commercial endoscope, the surgeon may pull the connecting member 300 with a small force.

In addition, since the friction force of the connecting member 300 at the connection point 130 is relieved, the shape deformation of the overtube 100 due to friction between the connecting member 300 and the overtube 100 may be mitigated.

FIG. 22 is a cross-sectional view illustrating the distal part 102 of the overtube 100 of the surgical tool transferring apparatus 10 according to an embodiment of the present disclosure and illustrating the first pulley 141 that protrudes from the distal part 102, and the second pulley 142 and a third pulley 143 that are disposed inside the overtube 100.

As illustrated in FIG. 22, a portion of the connection point 130 may protrude from the distal part 102 of the overtube 100, and a remaining portion of the connection point 130 may be positioned inside the overtube 100.

The first pulley 141, the second pulley 142, and the third pulley 143 are coupled to the overtube 100 such that the first pulley 141, the second pulley 142, and the third pulley 143 are capable of being rotated, and the first pulley 141, the second pulley 142, and the third pulley 143 disposed at portions where the movement direction of the connecting member 300 is changed at the connection point 130, thereby being capable of supporting the connecting member 300 such that the first pulley 141, the second pulley 142, and the third pulley 143 are capable of being rotated.

Therefore, since the friction force of the connecting member 300 at the connection point 130 is relieved, the energy required for driving the second actuator 500 may be reduced. In the commercial endoscope, the surgeon may pull the connecting member 300 with a small force.

In addition, since the friction force of the connecting member 300 at the connection point 130 is relieved, the shape deformation of the overtube 100 due to friction between the connecting member 300 and the overtube 100 may be mitigated.

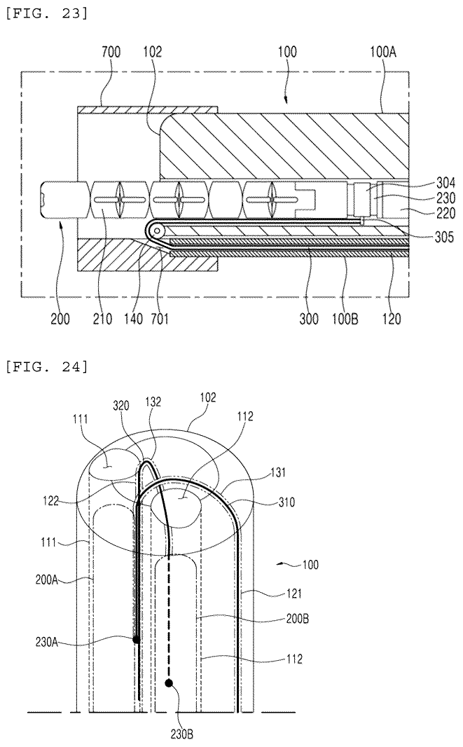

FIG. 23 is a cross-sectional view illustrating the distal part 102 of the overtube 100 of the surgical tool transferring apparatus 10 according to an embodiment of the present disclosure and illustrating the cap 700 and the connection point 130 of the distal part 102.

As illustrated in FIG. 23, the overtube 100 may include a main tube 100A and an auxiliary tube 100B. The main tube 100A forms the tool channel 110. The main tube 100A may be formed as an insertion section of a conventional endoscope.

The auxiliary tube 100B may be attached to an outer surface of the main tube 100A and may form the connecting member channel 120 along a longitudinal direction of the main tube 100A. The auxiliary tube 100B has a long tube shape. The auxiliary tube 100B is a portion that enters the intraluminal space of the human body together with the main tube 100A through natural openings such as the patient's mouth, anus, vagina, urethra, and so on.

The auxiliary tube 100B is inserted into the lumen together with the main tube 100A, and may be flexibly bent according to a curve in the lumen. The auxiliary tube 100B may be manufactured from an elastically deformable material such as medical-grade rubber, silicone, or the like.

As illustrated in FIG. 23, the connection point 130 is disposed between the tool channel 110 and the auxiliary tube 100B. The connection point 130 may protrude further from the distal part 102 of the overtube 100 than the tool channel 110. The pulley 140 is coupled to the overtube 100 such that the pulley 140 is capable of being rotated, and the pulley 140 may support the connecting member 300 at the connection point 130 such that the pulley 140 is capable of being rotated.

The cap 700 is coupled to a tip end (distal part 102) of the main tube 100A. In this state, the cap 700 covers a tip end of the auxiliary tube 100B.

The cap 700 forms a connection passage 701 in an inner surface of the cap 700. The connection passage 701 forms a passage connecting the connection point 130 of the main tube 100A to the connecting member channel 120. That is, the cap 700 forms a portion of the connection point 130.

Therefore, even when the overtube 100 including the connecting member channel 120 is not newly manufactured, the surgical tool 200 may be quickly and smoothly moved from the tool channel 110 toward the distal part 102 of the overtube 100 by attaching the auxiliary tube 100B to the insertion section of the conventional endoscope and pulling the connecting member 300 outside the human body.

FIG. 24 is a transparent perspective view illustrating the distal part 102 of the overtube 100 of the surgical tool transferring apparatus 10 according to an embodiment of the present disclosure and illustrating the first surgical tool 200A, the second surgical tool 200B, the first connecting member 310, and the second connecting member 320.

As illustrated in FIG. 24, the surgical tool 200 may include the first surgical tool 200A and the second surgical tool 200B, and the tool channel 110 may include the first tool channel 111 and the second tool channel 112. The first surgical tool 200A is inserted into the first tool channel 111, and the second surgical tool 200B is inserted into the second tool channel 112.

The connecting member 300 may include the first connecting member 310 and the second connecting member 320, and the connecting member channel 120 may include the first connecting member channel 121 and the second connecting member channel 122.

The connection point 130 may include the first connection point 131 and the second connection point 132. The first connection point 131 connects the first tool channel 111 and the first connecting member channel 121 to each other. The second connection point 132 connects the second tool channel 112 and the second connecting member channel 122 to each other.

The coupling part 230 may include the first coupling part 230A and the second coupling part 230B. The first coupling part 230A is disposed on the first surgical tool 200A, and the first connecting member 310 is connected to the first coupling part 230A. The second coupling part 230B is disposed on the second surgical tool 200B, and the second coupling member 320 is connected to the second coupling part 230B.

Therefore, the first connecting member 310 is inserted into the first connecting member channel 121, and is connected to the first coupling part 230A through the first connection point 131. The second connecting member 320 is inserted into the second connecting member channel 122, and is connected to the second coupling part 230B through the second connection point 132.

The first connection point 131 and the second connection point 132 may connect the tool channel 110 and the connecting member channel 120 to each other. At this time, in order for the first connection point 131 and the second connection point 132 do not overlap each other, the first connection point 131 and the second connection point 132 may be spaced apart from each other along the longitudinal direction of the overtube 100.

According to the present disclosure, when the connecting member 300 is pulled from outside the human body, the surgical tool 200 is moved in the tool channel 110 such that the coupling part 230 is positioned adjacent to the connection point 130, so that the surgical tool transferring apparatus 10 capable of moving the surgical tool 200 inserted into the tool channel 110 of the overtube 100 rapidly and smoothly to the affected region inside the human body even when a bend occurs in the overtube 100 may be provided.

Although the specific embodiment of the present disclosure has been described and illustrated above, it is apparent to those skilled in the art that the present disclosure is not limited to the embodiment disclosed herein and various modifications and changes can be made without departing from the spirit and scope of the present disclosure. Therefore, such modifications and changes should not be individually construed from the spirit or point of view of the present disclosure, and it should be understood that modified embodiments belong to the claims of the present disclosure.

(Description of Reference Numerals)

| | |
|---|---|
| 10: Surgical tool transferring apparatus | |
| 100: Overtube | 200: Surgical tool |
| 100A: Main tube | 200A: First surgical tool |
| 100B: Auxiliary tube | 200B: Second surgical tool |
| 101: Proximal part | 210: Bending part |
| 102: Distal part | 220: Flexible tube |
| 110: Tool channel | 230: Coupling part |
| 111: First tool channel | 230A: First coupling part |
| 112: Second tool channel | 230B: Second coupling part |
| 120: Connecting member channel | 231: Coupling body |
| 121: First connecting member | 232: Boundary body |
| channel | |
| 122: Second connecting member | 233: Connecting member fixing |
| channel | part |
| 123: Third connecting member | 234: Coupling groove |
| channel | |
| 130: Connection point | 300: Connecting member |
| 131: First connection point | 301: Connecting member fixing |
| | part |
| 132: Second connection point | 302: Snare portion |
| 133: Third connection point | 303: Sphere |
| 134: Fourth connection point | 304: Bistable band |
| 140: Pulley | 305: Connecting member fixing |
| | part |
| 141: First pulley | 310: First connecting member |
| 142: Second pulley | 320: Second connecting member |
| 143: Third pulley | 330: Third connecting member |
| 150: Camera | 340: Fourth connecting member |
| 400: First actuator | 800: Roll feeder |
| 500: Second actuator | 810: Rotation roller |
| 600: Manipulating device | 820: Interlocking pulley |
| 700: Cap | 701: Connection passage |

1. A surgical tool transferring apparatus comprising:
an overtube which is flexible enough to be inserted into a subject and which comprises a tool channel and a connecting member channel;
a surgical tool inserted into the tool channel; and
a connecting member inserted into the connecting member channel and connected to a coupling part of the surgical tool by passing through a connection point between the tool channel and the connecting member channel,
wherein the surgical tool is moved in the tool channel such that the coupling part is positioned adjacent to the connection point when the connecting member is pulled.

2. The surgical tool transferring apparatus of claim 1, wherein the surgical tool comprises:
a bending part configured to be bent by manipulating a manipulating device; and
flexible tube connecting the bending part and the manipulating device to each other,
wherein the coupling part is disposed on one side of the flexible tube or one side of the bending part.

3. The surgical tool transferring apparatus of claim 1, wherein the surgical tool comprises:
a bending part configured to be bent by manipulating a manipulating device; and
a flexible tube connecting the bending part and the manipulating device to each other,
wherein the coupling part is interposed between the bending part and the flexible tube.

4. The surgical tool transferring apparatus of claim 1, wherein a connecting member fixing part that protrudes toward an inner surface of the tool channel is formed on the coupling part, and the connecting member is coupled to the connecting member fixing part.

5. The surgical tool transferring apparatus of claim 1, wherein the connecting member is coupled to a connecting member fixing part, and a coupling groove into which the connecting member fixing part is inserted such that the connecting member fixing part is capable of being attached to and detached from the coupling groove is formed in an outer surface of the coupling part.

6. The surgical tool transferring apparatus of claim 1, wherein an end portion of the connecting member forms a snare portion such that the snare portion is configured to tighten an outer surface of the coupling part by a tension of the connecting member.

7. The surgical tool transferring apparatus of claim 6, wherein a plurality of spheres is threaded on the snare portion of the connecting member.

8. The surgical tool transferring apparatus of claim 6, wherein the coupling part comprises:
a coupling body tightened by the snare portion; and
a boundary body positioned on both ends of the coupling body with respect to a direction in which the surgical tool is moved in the tool channel, the boundary body being positioned closer to an inner surface of the tool channel than the coupling body.

9. The surgical tool transferring apparatus of claim 1, wherein the connecting member is coupled to a bistable band having a bistable structure, and the bistable band is coupled to an outer surface of the coupling part such that the bistable band is capable of being attached to and detached from the outer surface of the coupling part.

10. The surgical tool transferring apparatus of claim 1, wherein the connecting member channel comprises a first connecting member channel and a second connecting member channel,
wherein the connection point comprises:
a first connection point connecting the tool channel and the first connecting member channel to each other; and
a second connection point connecting the tool channel and the second connecting member channel to each other, and
wherein the connecting member comprises:
a first connecting member inserted into the first connecting member channel and connected to the coupling part through the first connection point; and
a second connecting member inserted into the second connecting member channel and connected to the coupling part through the second connection point.

11. The surgical tool transferring apparatus of claim 10, wherein the tool channel comprises a first tool channel and a second tool channel,
wherein the surgical tool comprises:
a first surgical tool inserted into the first tool channel; and
a second surgical tool inserted into the second tool channel,
wherein the first connection point connects the first tool channel and the first connecting member channel to each other, and the second connection point connects the second tool channel and the second connecting member channel to each other, and
wherein the coupling part comprises:
a first coupling part which is disposed on the first surgical tool and to which the first connecting member is connected; and
a second coupling part which is disposed on the second surgical tool and to which the second connecting member is connected.

12. The surgical tool transferring apparatus of claim 1, wherein the tool channel and the connecting member channel are opened at a tip end of the overtube, and the connection point is provided on the tip end of the overtube.

13. The surgical tool transferring apparatus of claim 1, wherein the connection point connects the tool channel and the connecting member channel to each other within the overtube.

14. The surgical tool transferring apparatus of claim 1, wherein a pulley is coupled to the overtube such that the pulley is capable of being rotated, and the pulley supports the connecting member at the connection point such that the pulley is capable of being rotated.

15. The surgical tool transferring apparatus of claim 1, wherein the overtube comprises:
a main tube forming the tool channel; and
an auxiliary tube attached to the main tube such that the connecting member channel is formed along a longitudinal direction of the main tube.

16. The surgical tool transferring apparatus of claim 15, wherein a cap is coupled to a tip end of the main tube, and the cap forms a portion of the connection point.

17. The surgical tool transferring apparatus of claim 1, further comprising:
a roll feeder configured to move the surgical tool by a rotational force of a rotation roller,
wherein the connecting member is interlocked with the rotation roller such that the connecting member is wound on or unwound from a rotating interlocking pulley so that a movement distance of the connecting member and a movement distance of the surgical tool in the tool channel are the same.
